SEQUENCE LISTING (14 sequences)

SEQ ID NO: 1 - DNA, 19 bp, Chikungunya virus
ctcttaccgggtttgttgc

SEQ ID NO: 2 - DNA, 18 bp, Chikungunya virus
gcctggacacctttcgac

SEQ ID NO: 3 - DNA, 45 bp, Chikungunya virus
aattctaatacgactcactataggggctcttaccgggtttgttgc

SEQ ID NO: 4 - DNA, 19 bp, Chikungunya virus
ctctcaggcaccatctggc

SEQ ID NO: 5 - DNA, 33 bp, Chikungunya virus
cgagcgactctcaggcaccatctggctcgctcg

SEQ ID NO: 6 - DNA, 20 bp, Chikungunya virus
gctcttaccgggtttgttgc

SEQ ID NO: 7 - DNA, 18 bp, Chikungunya virus
gcctggacacctttcgac

SEQ ID NO: 8 - DNA, 21 bp, Chikungunya virus
actctcaggcaccatctggct

SEQ ID NO: 9 - DNA, 21 bp, Chikungunya virus
ccaaattgtccyggtcttcct

SEQ ID NO: 10 - DNA, 22 bp, Chikungunya virus
aagctycgcgtcctttaccaag

SEQ ID NO: 11 - DNA, 24 bp, Chikungunya virus
ccaatgtcytcmgcctggacacct

SEQ ID NO: 12 - DNA, 48 bp, Chikungunya virus
aattctaatacgactcactataggggcccaaattgtccyggtcttcct

SEQ ID NO: 13 - DNA, 22 bp, Chikungunya virus
aagctycgcgtcctttaccaag

SEQ ID NO: 14 - DNA, 38 bp, Chikungunya virus
cgagcgacccaatgtcytcmgcctggacacctcgctcg

(19) United States
(12) Patent Application Publication
(10) Pub. No.: US 2010/0216117 A1
(43) Pub. Date: Aug. 26, 2010

(54) OLIGONUCLEOTIDES, USE, METHOD OF DETECTION AND KIT FOR DIAGNOSING THE PRESENCE OF THE CHIKUNGUNYA VIRUS E1 GENE

(75) Inventor: Jean-Noel Telles, Lyon (FR)

Correspondence Address: OLIFF & BERRIDGE, PLC, P.O. BOX 320850, ALEXANDRIA, VA, 22320-4850, US

(73) Assignee: BIOMERIEUX, Marcy L'Etoile (FR)

(21) Appl. No.: 12/673,688

(22) PCT Filed: Sep. 11, 2008
(86) PCT No.: PCT/FR08/51625

§ 371 Date: Feb. 16, 2010

(30) Foreign Application Priority Data
Sep. 14, 2007 (FR) 0757598

(51) Int. Cl. C12Q 1/70; C07H 21/04; C12Q 1/68

(52) U.S. Cl. 435/5; 536/23.1; 536/24.1; 536/24.32; 435/6

(57) ABSTRACT

The present invention concerns oligonucleotides intended to enable the amplification and the detection of a target sequence located in the E1 gene of the Chikungunya virus. These oligonucleotides are between 10 and 50 nucleotides in length and comprise at least one fragment of 10 consecutive nucleotides derived from the following sequences: SEQ ID No. 1: 5′-CTCTTACCGGGTTTGTTGC-3′ or SEQ ID No. 2: 5′-GCCTGGACACCTTTCGAC-3′, or the sequence complementary thereto. The invention also concerns the oligonucleotide which enables detection of the amplicons, the use of these oligonucleotides, a method of detection and a kit for diagnosing the presence of the E1 gene of the Chikungunya virus. The invention has a preferred use in the diagnostics field.

The present invention relates to oligonucleotides intended to enable amplification and detection of a target sequence located in the Chikungunya virus E1 gene. The invention also relates to the use of these oligonucleotides, a method of detection and a kit for diagnosing the presence of the Chikungunya virus E1 gene.

The Chikungunya virus is an arbovirus of the alphavirus genus which is transmitted by Aedes mosquitoes. It was isolated for the first time in 1953. Since then, Chikungunya epidemics have occurred in tropical Africa and in Asia. An unprecedented epidemic occurred on Reunion Island, which has 775 000 inhabitants and for which more than 244 000 cases were reported on 20 Apr. 2006.

At the current time, there is no routine diagnosis for Chikungunya infection and very few laboratories have the ability to diagnose this virus. The techniques used in these reference laboratories are the following:
serological testing using common techniques (haemaglutination inhibition, complement fixing, immunofluorescence, ELISA),
amplification and detection by real-time RT-PCR. The serological testing is based on the detection of IgM and IgG antibodies which appear at various times of the infection. The IgMs are detected in the serum on average 5 days after the onset of the clinical signs and persist for several weeks to three months. The IgGs are identified by taking two samples (acute phase and convalescence), and persist for years.
   The sensitivity and the specificity of these tests are not well established, in particular the possibility of false positives due to cross reactions with IgMs for dengue or for other arboviruses.
   In parallel, real-time RT-PCR amplification tests have been developed. The value of molecular biology in the diagnosis of infectious diseases is well known. It in fact enables rapid detection of the virus genome, from the initial viraemic phase onwards, said phase corresponding to the first seven days in the case of an infection with the Chikungunya virus.
   Thus, Pastorino et al. describe an RT-PCR test for the real-time detection of the Chikungunya virus using the TaqMan® method (Journal of Virological Methods, 124 (2005) 65-71). The amplification primers and the detection probe were selected in the region of the gene of the E1 structural protein. The sensitivity obtained is 1.2×10⁻² infective dose per reaction. In another publication, Parida et al. describe the detection of the Chikungunya virus by means of an alternative method to PCR, known as RT-LAMP for “reverse transcription loop-mediated isothermal amplification” (J. Clin. Microbiol. 2007 February; 45(2):351-7). The primers were selected in the gene of the E1 protein. A sensitivity of 20 transcript copies per reaction was determined.
   The drawback of this technique is that it requires 6 different primers in order to carry out the amplification reaction. In addition, the detection is not carried out with a specific probe, but by turbidimetry. Thus, when the reaction is not specific and another virus is amplified, a false positive will be detected.

Thus, a rapid, specific and very sensitive test for identifying the Chikungunya virus is still awaited.

The present invention therefore relates to a pair of oligonucleotides intended to enable the amplification of a target sequence located in the E1 gene of the Chikungunya virus genome, the pair of oligonucleotides consisting of:
a first oligonucleotide between 10 and 50 nucleotides long and comprising at least one fragment of 10 consecutive nucleotides derived from:
SEQ ID No. 1: 5′-CTCTTACCGGGTTTGTTGC-3′, or the sequence complementary thereto, and
a second oligonucleotide between 10 and 50 nucleotides long and comprising at least one fragment of 10 consecutive nucleotides derived from:
SEQ ID No. 2: 5′-GCCTGGACACCTTTCGAC-3′, or the sequence complementary thereto.

In one particular embodiment of the invention, the first oligonucleotide additionally comprises a promoter sequence which can be recognized by a DNA-dependent RNA polymerase enzyme. Preferably, the promoter sequence which can be recognised by a DNA-dependent RNA polymerase enzyme is a T7 polymerase.

Thus, when this oligonucleotide, to which the promoter sequence is added, enables the amplification of the target sequence located in the E1 gene, it consists essentially of the following sequence:

SEQ ID No. 3: 5′-ATTCTAATACGACTCACTATAGGGGCTCTTACCGGGTTTGTTGC-3′. The part of the sequence in italics corresponds to the T7 promoter sequence. The sequence underlined corresponds to a sequence which creates a space, termed linker, between the promoter sequence and the oligonucleotide of sequence SEQ ID No. 1.

The invention also relates to an oligonucleotide intended to be used as a probe for detecting a target sequence located in the E1 gene of the Chikungunya virus genome, the detection probe being between 10 and 50 nucleotides long and comprising at least one fragment of 10 consecutive nucleotides derived from:

SEQ ID No. 4: 5′-CTCTCAGGCACCATCTGGC-3′, or the sequence complementary thereto, said sequence comprising at least one labelling means.

In one advantageous embodiment of the invention, the oligonucleotide used as a detection probe enables real-time detection and is flanked by two arms, one in the 5′ position and the other in the 3′ position of said oligonucleotide, each arm being between 5 and 15 nucleotides, preferably between 6 and 10 nucleotides, in length and these arms being complementary to one another in order to present the oligonucleotide in the form of a loop in the absence of the target sequence located in the E1 gene of the Chikungunya virus genome.

In one even more advantageous embodiment, the detection probe comprises a fluorescent label at the free end of one of the arms and a fluorescence quencher at the free end of the other arm. Preferably, the detection probe is constituted of a “molecular beacon”. Molecular beacons are detection probes in the form of single-stranded oligonucleotides, which have a stem loop structure well known to those skilled in the art. The loop contains a probe sequence complementary to the target sequence (amplicon in general), and the stem is formed by the hybridization of two sequences forming arms, which are each located at each end of the probe. A fluorophore is covalently bonded to the end of one of the two arms and a fluorescence quencher is covalently bonded to the end of the other arm. Molecular beacons do not fluoresce when they are free in solution, the loop structure maintaining the fluorophore and the quencher in proximity to one another, thereby leading to transfer of the fluorescence from the fluorophore to the quencher. Said fluorescence quencher is a nonfluorescent chromophore which dissipates the energy received from the fluorophore, as heat. However, in the presence of complementary amplicons, when they hybridize to these targets, they undergo a conformational change which enables them to fluoresce. In this case, there is formation of a probe-target hybrid which is longer and more stable than the hybrid created by the two arms of the stem. The rigidity and the length of the probe-hybrid hybrid prevents the simultaneous existence of the stem hybrid. Consequently, the molecular beacon undergoes a spontaneous conformational reorganization which forces the stem hybrid to dissociate and the fluorophore and the quencher to move away from one another, thereby restoring the fluorescence.

More specifically, the detection probe is constituted of a molecular beacon preferably constituted of:

SEQ ID No. 5: 5′-[6-FAM]-CGAGCGACTCTCAGGCACCATCTGGCTCGCTCG-[DabSyl]-3′, or the sequence complementary thereto. The sequence in italics corresponds to the arms mentioned above, constituting the stem.

According to the present invention, the two oligonucleotides intended to be used as amplification primers, and the oligonucleotide intended to be used as a detection probe, are each between 10 and 50 nucleotides long and comprise at least one fragment of 10 consecutive nucleotides derived from the sequences SEQ ID Nos. 2 and 4, respectively. Preferably, each oligonucleotide is between 12 and 30 nucleotides long and comprises at least one fragment of 10 or consecutive nucleotides, and even more preferably, each oligonucleotide is between 15 and 26 nucleotides long and comprises at least one fragment of 10, 12 or 15 nucleotides.

The present invention also proposes the use of the pair of oligonucleotides or of the probe as described above, in a reaction for amplifying nucleic acids of the genome of the Chikungunya virus that may be present in a biological sample.

The invention also relates to a method for detecting nucleic acids of the Chikungunya virus that may be present in a sample, in which the sample is subjected to a nucleic acid amplification reaction using a pair of oligonucleotides, as described above, in the presence of the amplification reagents necessary for such an amplification and where the presence of amplicons of interest is detected.

This detection method may be based on an RT-PCR amplification reaction.

Alternatively, this detection method may be based on a transcriptional amplification technique.

Preferably, this technique is the NASBA technique. The NASBA technique is a method of isothermal amplification of nucleic acids involving several enzyme activities, which enables rapid detection of the Chikungunya virus. The amplification of nucleic acids by this approach is very suitable for RNA genomes, by virtue of the presence of a step of reverse transcription directly in the amplification reaction.

Thus, the invention also relates to a method for amplifying the E1 gene of the Chikungunya virus that may be present in a sample, comprising the following steps:
incubating the sample in an amplification buffer in the presence of two amplification primers, each being between 10 and 50 nucleotides long, one additionally comprising a promoter sequence, the other having a polarity opposite to that of the primer associated with the promoter sequence, so as to hybridize respectively upstream and downstream of a region of interest located in the E1 gene of the Chikungunya virus,
adding the following reagents to the sample:
   an enzyme which has an RNA-dependent DNA polymerase activity,
   an enzyme which has a DNA-dependent DNA polymerase activity,
   an enzyme which has an RNase H activity,
   an enzyme which has a DNA-dependent RNA polymerase activity, and
maintaining the reaction mixture thus created under suitable conditions and for a period of time sufficient for an amplification to take place.

Four enzymes are listed above, but it is entirely possible to make use of an enzyme which has two or even three of the activities mentioned above; in this case, the use of three or even two enzymes remains possible and covered by the invention. In addition, other elements are necessary for establishing an amplification, such as nucleotides or else buffer solutions. These buffer solutions can be optimized as a function of the amplification technique used or of the oligonucleotides present in the reaction. In a transcriptional amplification reaction, such as a NASBA reaction, these buffer solutions may contain, for example, DMSO, which improves the amplification reaction (as described in document PCT/US90/04733). In addition, it is also possible to add an internal control to the amplification reaction, in order to prevent the presence of a false negative due to failure of the amplification process. The use of an internal control in a transcriptional amplification reaction is described in document PCT/EP93/02248. Such an internal control is selected in such a way that it does not compete with the target nucleic acid in the amplification reaction. All these elements are well known to those skilled in the art.

Finally, the invention proposes a kit for detecting the E1 gene of the Chikungunya virus that may be present in a sample, containing:
at least one pair of oligonucleotides as described above for carrying out the amplification of the E1 gene,
at least one oligonucleotide which is labelled or which can be labelled, as described above, and which has a nucleic acid sequence substantially complementary to at least one part of the nucleic acid sequence amplified,
reagents necessary for carrying out an amplification reaction.

Preferably, the reagents necessary for carrying out an amplification reaction are reagents for a NASBA amplification.

The term “substantially complementary” is intended to mean that hybridization occurs between an oligonucleotide which is labelled or which can be labelled, otherwise known as amplification primer or detection probe, and at least one part of the target or amplified nucleic acid sequence, termed amplicon, this hybridization being specific and selective so as to enable the amplification or the detection of the amplicon of interest.

The term “detectable label” is intended to mean at least one label capable of directly generating a detectable signal. For example, the presence of biotin is considered to be direct labelling, since it is detectable, even though it is possible to subsequently associate it with labelled streptavidin. A nonlimiting list of these labels follows:
enzymes which produce a signal that is detectable, for example, by colorimetry, fluorescence or luminescence, for instance horseradish peroxidase, alkaline phosphatase, β-galactosidase, glucose-6-phosphate dehydrogenase,
chromophores, for instance fluorescent or luminescent compounds, colorants,
electron-dense groups detectable by electron microscopy or by virtue of their electrical property, for instance conductivity, amperometry, voltametry, impedance,
detectable groups, for example the molecules of which are of sufficient size to induce detectable modifications of their physical and/or chemical characteristics, this detection can be carried out by optical methods such as diffraction, surface plasmon resonance, surface variation, contact angle variation or physical methods such as atomic force spectroscopy or tunnel effect,
radioactive molecules such as ³²P, ³⁵S or ¹²⁵I.

In one particular embodiment of the present invention, the label is electrochemically detectable, and in particular the label is a derivative of an iron complex, such as a ferrocene.

The term “nucleic acid” signifies a series of at least two deoxyribonucleotides or ribonucleotides optionally comprising at least one modified nucleotide, for example at least one nucleotide comprising a modified base, such as inosine, methyl-5-deoxycytidine, dimethylamino-5-deoxyuridine, deoxyuridine, 2,6-diaminopurine, bromo-5-deoxyuridine or any other modified base enabling hybridization. This polynucleotide may also be modified at the level of the internucleotide bond, for instance phosphorothioates, H-phosphonates or alkyl phosphonates, or at the level of the backbone, for instance alpha-oligonucleotides (FR 2 607 507) or PNAs (M. Egholm et al., J. Am. Chem. Soc., 114, 1895-1897, 1992) or 2′-O-alkyl riboses and LNAs (B W, Sun et al., Biochemistry, 4160-4169, 43, 2004). The nucleic acid may be natural or synthetic, an oligonucleotide, a polynucleotide, a nucleic acid fragment, a ribosomal RNA, a messenger RNA, a transfer RNA, or a nucleic acid obtained by means of an enzymatic amplification technique such as:
PCR (Polymerase Chain Reaction), described in U.S. Pat. No. 4,683,195, U.S. Pat. No. 4,683,202 and U.S. Pat. No. 4,800,159, and its derivative RT-PCR (Reverse Transcription PCR), in particular in a one-step format, as described in patent EP-B-0.569.272,
LCR (Ligase Chain Reaction), disclosed, for example, in patent application EP-A-0.201.184,
RCR (Repair Chain Reaction), described in patent application WO-A-90/01069,
3SR (Self Sustained Sequence Replication) with patent application WO-A-90/06995,
NASBA (Nucleic Acid Sequence-Based Amplification) with patent application WO-A-91/02818,
TMA (Transcription Mediated Amplification) with U.S. Pat. No. 5,399,491, and
RCA (Rolling Circle Amplification (U.S. Pat. No. 6,576,448)).
   The term “amplicons” is therefore used to denote the nucleic acids generated by an enzymatic amplification technique.
   Each of these modifications may be taken in combination.

The amplification and detection steps disclosed above may be preceded by a purification step. The term “purification step” is intended to mean in particular the separation of the nucleic acids of the microorganisms from the cellular constituents released in the lysis step which precedes the nucleic acid purification. These lysis steps are well known and, by way of exemplary indication, use may be made of the lysis methods as described in patent applications:

WO-A-00/60049 on lysis by sonication,

WO-A-00/05338 on mixed magnetic and mechanical lysis,

WO-A-99/53304 on electrical lysis, and

WO-A-99/15621 on mechanical lysis.

Those skilled in the art may use other well known methods of lysis such as heat shock or osmotic shock or treatments with chaotropic agents, such as guanidinium salts (U.S. Pat. No. 5,234,809).
This step generally makes it possible to concentrate the nucleic acids. By way of example, it is possible to use solid supports, such as magnetic particles (in this respect see U.S. Pat. No. 4,672,040 and U.S. Pat. No. 5,750,338) and thus to purify the nucleic acids, which are attached to these magnetic particles, by means of a washing step. This nucleic acid purification step is particularly advantageous if it is desired to subsequently amplify said nucleic acids. A particularly advantageous embodiment of these magnetic particles is described in patent applications WO-A-97/45202 and WO-A-99/35500.

The term “solid support” as used herein includes all the materials to which a nucleic acid can be attached. Synthetic materials or natural materials, which have optionally been chemically modified, may be used as a solid support, in particular polysaccharides, such as cellulose-based materials, for example paper, cellulose derivatives, such as cellulose acetate and nitrocellulose, or dextran; polymers, copolymers, in particular based on monomers of the styrene type, natural fibres such as cotton, and synthetic fibres, such as nylon; inorganic materials, such as silica, quartz, glasses, ceramics; lattices; magnetic particles; metal derivatives, gels, etc. The solid support may be in the form of a microtitration plate, a membrane, a particle or a substantially flat plate made of glass or silicon or derivatives.

The entire protocol (from the sample taken to the detection of the amplicons) can be carried out in one and the same container or tube, processed manually or in an automated device.

The figures and the example attached represent one particular embodiment and cannot be considered to limit the scope of the present invention.

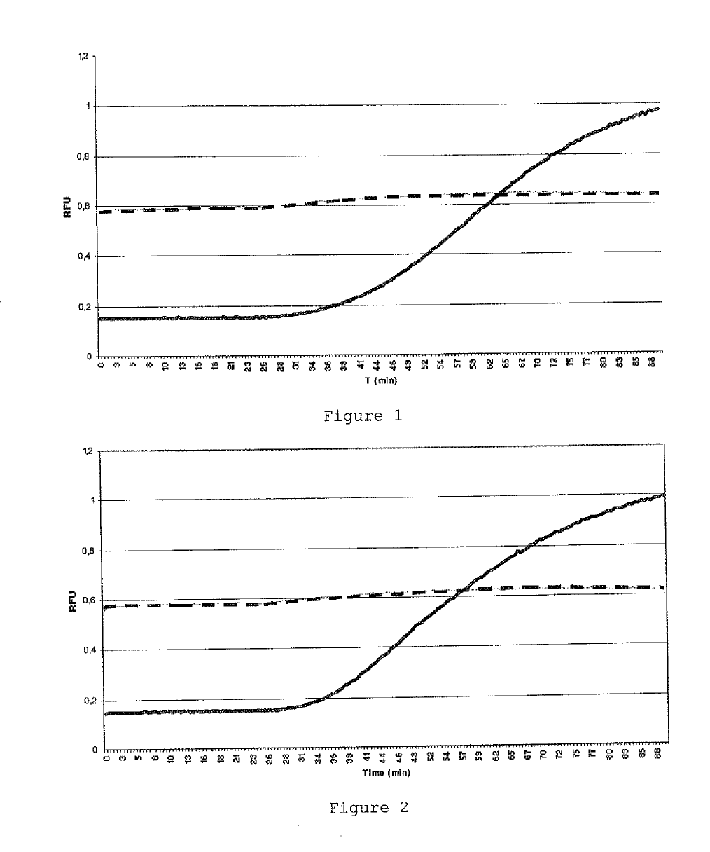

FIGS. 1 to 8: Detection of the Chikungunya virus at various concentrations in relative fluorescence units (RFU along the y-axis) per unit of time (T along the x-axis). The dashed-line curve corresponds to the signal emitted by the probe, according to the invention, complementary to a sequence of the Chikungunya virus, and the solid-line curve corresponds to the internal control, said control being a synthetic transcript of approximately 1000 bases in size which encompasses the region amplified by the NASBA primers; it will therefore be coamplified with the wild-type sequence present in the sample. However, this internal control has been modified so as to replace the region where the detection probe normally binds, with a sequence which does not correspond to Chikungunya. This modified region is recognized by the detection probe for the internal control.

FIG. 1: negative plasma

FIG. 2: positive plasma, 4.18 copies/ml

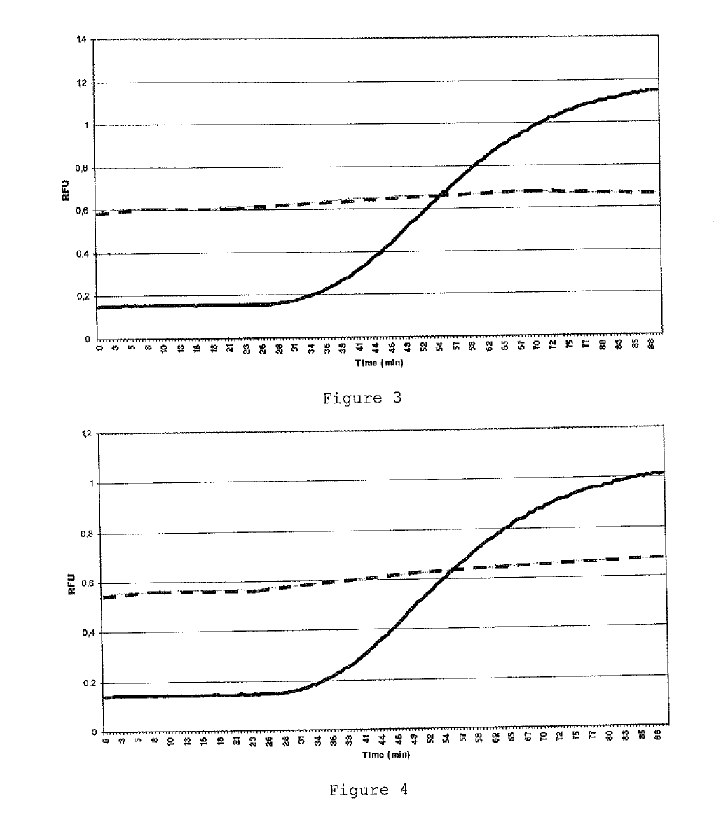

FIG. 3: positive plasma, 41.8 copies/ml

FIG. 4: positive plasma, 418 copies/ml

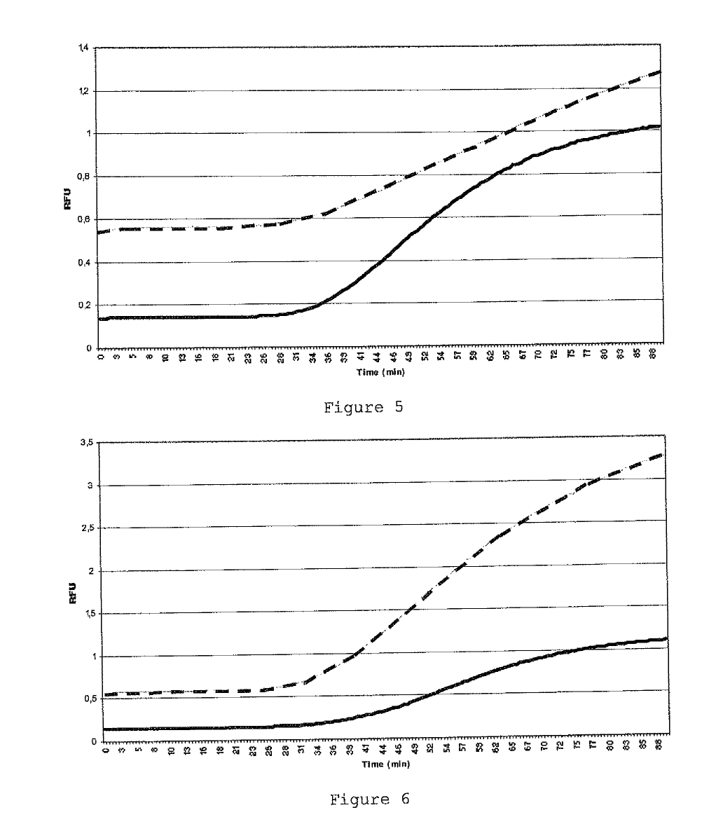

FIG. 5: positive plasma, 4180 copies/ml

FIG. 6: positive plasma, 41 800 copies/ml

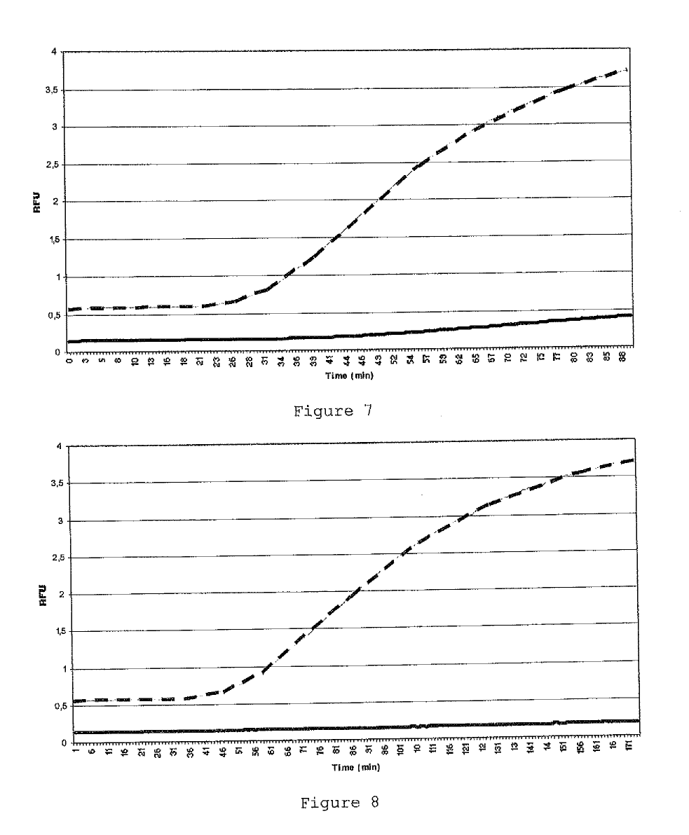

FIG. 7: positive plasma, 418 000 copies/ml

FIG. 8: positive plasma, 4 180 000 copies/ml

### EXAMPLE 1

### Experiment to Evaluate the Primers and the Detection Probe for the Chikungunya Virus on an RNA Originating from a Clinical Sample from Reunion

The sequences of the pair of oligonucleotides (P1 and P2) and the detection probe (MB) present in the form of a molecular beacon, are indicated below:

Sequences of the primers and probe used:
P1, SEQ ID NO. 3:
5′- [symbol] [symbol] [symbol] CTCTTACCGGGTTTGT
TGC-3′
P2, SEQ ID NO. 2:
5′-GCCTGGACACCTTTCGAC-3′
MB, SEQ ID NO. 5:
5′-CGAGCGACTCTCAGGCACCATCTGGCTCGCTCG-3′

The sequence indicated in bold and in italics corresponds to the T7 promoter sequence, recognized by the T7 RNA polymerase, and is found in the P1 oligonucleotides for implementation of the NASBA technique. The sequence underlined corresponds to a sequence which creates a space, termed linker, between the promoter sequence and the oligonucleotide of sequence SEQ ID No. 1. The sequences doubly underlined correspond to the sequences forming the stem of the molecular beacon.

### Procedure:

Source of the Chikungunya Virus:

Dilutions of plasmas from infected and quantified patients, originating from the Centre Hospitaller Sud [Hospital Centre South] St Denis, Reunion.

Nucleic Acid Extraction:

The nucleic acids were extracted using the easyMAG system (bioMérieux B.V., Boxtel, Holland) with the NucliSENS Magnetic Extraction Reagents (bioMérieux B.V., Boxtel, Holland, NucliSENS EasyMAG extraction buffer 1 #280130, NucliSENS EasyMAG extraction buffer 2 #280131, NucliSENS EasyMAG extraction buffer 3 #280132, NucliSENS EasyMAG magnetic silica #280133, NucliSENS EasyMAG lysis buffer #280134). The extractions were carried out on 200 μl of each of the plasma dilutions. The dilutions correspond to concentrations of 41 copies/ml, 418 copies/ml, 4180 copies/ml, 41 800 copies/ml, 418 000 copies/ml, 4 180 000 copies/ml.

Amplification/Detection:

According to the instructions of the NucliSENS EasyQ Basic Kit V2 (bioMérieux B.V., Boxtel, Holland), a single reaction mixture enabling the simultaneous detection of the Chikungunya E1 gene and of the internal control is prepared. Briefly, 11 μl of water, 13 μl of KCl at 1.2M, 4 μl of each of the oligonucleotides at 10 μM, and 0.8 μl of each of the molecular beacons at 20 μM (one molecular beacon specific for Chikungunya and one probe specific for the internal control) were added to 64 μl of diluent. A volume of 10 μl of the mixture was then added to 5 μl of the solutions of the previously extracted RNAs.

### Results:

The results can be seen on FIGS. 1 to 8 and show that the test works and detects a positive Chikungunya plasma down to the concentration of 4180 copies/ml (equivalent to approximately 150 copies of Chikugunya RNA/NASBA reaction). It is observed that, the higher the number of Chikungunya RNA copies, the greater the amplitude of the curve.
The results also show that the internal control is correctly detected and properly plays its role of validation of the test. The internal control signal decreases proportionally to the number of copies of Chikungunya RNA. The internal control signal is at its maximum in the negative plasma, and is minimal in the presence of a high number of copies of Chikungunya RNA. This is because, given that the amplification is a coamplification, the amount of the internal control was calculated in such a way that the internal control does not compete too much with the wild-type sequence of the virus. Thus, the internal control is normally amplified when it is alone (negative sample), but as soon as a wild-type Chikungunya sequence is present, it is this sequence which is amplified, going as far as complete inhibition of the amplification of the internal control when the wild-type sequence is present at a high concentration. Thus, in order for the test to be validated, there must always be a signal: if the sample is negative, there is the positive internal control, if the sample is positive, there is a positive signal with or without a signal for the internal control (depending on whether or not the sample is strongly positive).

### EXAMPLE 2

### Comparison of the Efficiency of the Amplification Primers and of the Detection Probe for the Chikungunya Virus with Respect to the Primers and the Probe of the RT-PCR Reference Technique

The primers and probe of the present invention and of the reference technique were compared by NASBA and by PCR, over a range of dilutions of quantified Chikungunya RNAs. The primers/probe of the reference technique were adapted in NASBA and those of the present invention were adapted to the reference technique.

### Procedure:

Source of Chikungunya RNA:

The experiments were carried out on a range of dilutions of purified RNAs from a source of Chikungunya originating from the INTSSA (Institut de Medecine Tropicale du Service de Santé des Armées [Institute of Tropical Medicine of the Armed Forces Health Department]).

Amplification/Detection:
NASBA:
   According to the instructions of the NucliSENS EasyQ Basic Kit V2 (bioMérieux B.V., Boxtel, Holland, batch 010712), a single reaction mixture for the amplification and the detection of the E1 gene of the Chikungunya virus is prepared. Briefly, 11 μl of water, 13 μl of KCl at 1.2M, 4 μl of each of the oligonucleotides at 10 μM, and 0.8 μl of the molecular probe at 20 μM were added to 64 μl of diluent. A volume of 10 μl of the mixture was then added to 5 μl of the solutions of the previously extracted RNAs. After incubation for 2 min at 65° C. and then for 2 min at 41° C., 5 μl of the enzyme mixture is added. The amplification and the real-time detection of the signal are carried out using the NucliSENS EasyQ Analyzer system (bioMérieux, Boxtel, Holland, reference 285060) for 90 min at 41° C. The results are analyzed using the NucliSENS EasyQ Director 2.0 software.
RT-PCR:
   The one-step RT-PCR was carried out in a final volume of 20 μl containing 2.5 μl of extracted Chikungunya RNA, 10 μl of 2× Thermoscript Reaction Mix Buffer (Invitrogen, batch 1207045, Germany), 2 pmol of Taqman probe, 9 pmol of each of the amplification primers, 25 mM MgSO₄, and 0.4 μl of the One Step RT-PCR enzyme mix (Invitrogen, batch 271375, Germany). The amplifications were carried out in a LightCycler instrument (Roche, Molecular Biochemicals, Germany) using the following parameters: 50° C. for 20 min, 95° C. for 2 min and 45 cycles with 95° C. for 5 sec, and 60° C. for 1 min.

### Primers and Probes Used:

1) Primers and probe of the present invention adapted to NASBA:

P1: SEQ ID NO. 3:
5′- [symbol] [symbol] [symbol] CTCTTACCGGGTTT
GTTGC-3′
P2: SEQ ID NO. 2:
5′-GCCTGGACACCTTTCGAC-3′
MB: SEQ ID NO. 5:
5′Fam-CGAGCGACTCTCAGGCACCATCTGGCTCGCTCG-
Dabsyl 3′.

2) Primers and probe of the invention adapted to an RT-PCR:

P1-CHIK-sansT7 [P1-CHIK-withoutT7]:
SEQ ID NO. 6:
5′-GCTCTTACCGGGTTTGTTGC-3′
P2: SEQ ID NO. 7:
5′-GCCTGGACACCTTTCGAC-3′
BEAC-CHIK-SANS-TIGE [BEAC-CHIK-WITHOUT-STEM]:
SEQ ID NO. 8:
5′Fam-ACTCTCAGGCACCATCTGGCT-Tamra3′.

3) Primers and probe derived from the publication of Pastorino et al. (the journal: Journal of Virological Methods, 124 (2005) 65-71) for an RT-PCR:

R-CHIK:
SEQ ID NO. 9:
5′-CCAAATTGTCCYGGTCTTCCT-3′
F-CHIK:
SEQ ID NO. 10:
5′-AAGCTYCGCGTCCTTTACCAAG-3′
P-CHIK:
SEQ ID NO. 11:
5′Fam-CCAATGTCYTCMGCCTGGACACCT-Tamra 3′

(the letter Y corresponding either to the base C or to the base T, and the letter M corresponding either to the base C or to the base A).
4) Primers and probe derived from the publication of Pastorino et al. (the journal: Journal of Virological Methods, 124 (2005) 65-71) adapted to a NASBA:

NASP1-PASTO-NAS:
SEQ ID NO. 12:
5′- [symbol] [symbol] [symbol] CCCAAATTGTCCYGGTCT
TCCT-3′
F-CHIC:
SEQ ID NO. 13:
5′-AAGCTYCGCGTCCTTTACCAAG-3′
BEAK-PASTO-NAS:
SEQ ID NO. 14:
5′ Fam-CGAGCGACCCAATGTCYTCMGCCTGGACACCTCGCTCG-
Dabsyl 3′.

### Results:

The results are summarized in the table below:

| | Primers and probe of | | Primers and probe derived | |
|---|---|---|---|---|
| | the present invention | | from the publication | |
| | (adapted in the case | | of Pastorino et al. | |
| Chikun- | of RT-PCR) | | (adapted in the case of NASBA) | |
| gunya | | RT-PCR | | RT-PCR |
| RNA | | Light Cycler** | | Light Cycler |
| dilution | NASBA* | (Ct) | NASBA | (Ct) |
| 10⁻⁴ | + | + | + | + |
| 10⁻⁵ | + | + | + | + |
| 10⁻⁶ | + | + | + | − |
| 10⁻⁷ | + | + | + | − |
| 10⁻⁸ | − | − | − | − |
| 10⁻⁹ | − | − | − | − |
| Negative | − | − | − | − |
| control | | | | |

*The NASBA test is positive when the fluorescence signal obtained exceeds the negative-control signal by 20%.
**The RT-PCR test is positive when a measurable Ct is obtained (the Ct corresponds to the number of cycles for which the signal becomes positive).

The results show that, over the Chikungunya RNA dilution range, the primers and probe of the present invention, used in NASBA, exhibit a better sensitivity than the primers and probe of the publication of Pastorino et al., used in the RT-PCR reference test. This is because the NASBA detects the 10⁻⁷ dilution, whereas the RT-PCR detects only to the 10⁻⁵ dilution. Furthermore, although, in NASBA, the various primers and probe exhibit an identical sensitivity, the primers and probe of the present invention can be used in RT-PCR and can increase the sensitivity by two logarithms compared with the sequences of Pastorino et al. in the reference method.

1. Pair of oligonucleotides intended to enable the amplification of a target sequence located in the E1 gene of the Chikungunya virus genome, the pair of oligonucleotides consisting of:
a first oligonucleotide between 10 and 50 nucleotides long and comprising at least one fragment of 10 consecutive nucleotides derived from:
SEQ ID No. 1: 5′-CTCTTACCGGGTTTGTTGC-3′, or the sequence complementary thereto, and
a second oligonucleotide between 10 and 50 nucleotides long and comprising at least one fragment of 10 consecutive nucleotides derived from:
SEQ ID No. 2: 5′-GCCTGGACACCTTTCGAC-3′, or the sequence complementary thereto.

2. Pair of oligonucleotides, according to claim 1, wherein the first oligonucleotide additionally comprises a promoter sequence which can be recognized by a DNA-dependent RNA polymerase enzyme.

3. Pair of oligonucleotides, according to claim 2, wherein the promoter sequence which can be recognized by a DNA-dependent RNA polymerase enzyme is a T7 polymerase.

4. Pair of oligonucleotides, according to claim 2, wherein the first oligonucleotide consists essentially of the following sequence:
SEQ ID No. 3:
5′-AATTCTAATACGACTCACTATAGGGGCTCTTACCGGGTTTGTTG
C-3′.

5. Oligonucleotide intended to be used as a probe for detecting a target sequence located in the E1 gene of the Chikungunya virus genome, the detection probe being between 10 and 50 nucleotides long and comprising at least one fragment of 10 consecutive nucleotides derived from:
SEQ ID No. 4: 5′-CTCTCAGGCACCATCTGGC-3′, or the sequence complementary thereto, said sequence comprising at least one labelling means.

6. Oligonucleotide according to claim 5, intended to enable real-time detection, wherein the oligonucleotide is flanked by two arms, one in the 5′ position and the other in the 3′ position of said oligonucleotide, each arm being between 5 and 15 nucleotides long, and these arms being complementary to one another in order to present the oligonucleotide in the form of a loop in the absence of the target sequence located in the E1 gene of the Chikungunya virus genome.

7. Oligonucleotide, according to claim 6, wherein the free end of one of the arms comprises a fluorescent label and the free end of the other arm comprises a fluorescence quencher.

8. Oligonucleotide, according to claim 6, wherein it consists essentially of the following sequence:
SEQ ID No. 5: 5′-[6-FAM]-CGAGCGACTCTCAGGCACCATCTGGCTCGCTCG-[DabSyl]-3′, or the sequence complementary thereto.

9. Pair of oligonucleotides, according to claim 1, wherein each oligonucleotide is between 12 and 30 nucleotides long and comprises at least one fragment of 10 or 12 consecutive nucleotides.

10. Method comprising utilizing a pair of oligonucleotides, according to claim 1, in a reaction for amplification of nucleic acids or utilizing an oligonucleotide as a probe for detection of the genome of the Chikungunya virus that may be present in a biological sample, the detection probe being between 10 and 50 nucleotides long and comprising at least one fragment of 10 consecutive nucleotides derived from:
SEQ ID No. 4: 5′-CTCTCAGGCACCATCTGGC-3′, or the sequence complementary thereto, said sequence comprising at least one labelling means.

11. Method for detecting nucleic acids of the Chikungunya virus that may be present in a sample, in which the sample is subjected to a nucleic acid amplification reaction using a pair of oligonucleotides, according to claim 1, in the presence of the amplification reagents necessary for such an amplification, and the presence of amplicons of interest is detected.

12. Method, according to claim 11, wherein the amplification reaction used is a RT-PCR.

13. Method, according to claim 11, wherein the amplification reaction used is a transcriptional amplification technique.

14. Method, according to claim 13, wherein the amplification reaction used is the NASBA technique.

15. Method for amplifying the E1 gene of the Chikungunya virus that may be present in a sample, comprising the following steps:
incubating the sample in an amplification buffer in the presence of two amplification primers, each being between 10 and 50 nucleotides long, one additionally comprising a promoter sequence, the other having a polarity opposite to that of the primer associated with the promoter sequence, so as to hybridize respectively upstream and downstream of a region of interest located in the E1 gene of the Chikungunya virus,
adding the following reagents to the sample:
an enzyme which has an RNA-dependent DNA polymerase activity,
an enzyme which has a DNA-dependent DNA polymerase activity,
an enzyme which has an RNase H activity,
an enzyme which has a DNA-dependent RNA polymerase activity, and
maintaining the reaction mixture thus created under suitable conditions and for a period of time sufficient for an amplification to take place.

16. Kit for detecting the E1 gene of the Chikungunya virus that may be present in a sample, containing:
at least one pair of oligonucleotides according to claim 1,
at least one oligonucleotide which is labelled or which can be labelled, the oligonucleotide being between 10 and 50 nucleotides long and comprising at least one fragment of 10 consecutive nucleotides derived from:
SEQ ID No. 4: 5′-CTCTCAGGCACCATCTGGC-3′, or the sequence complementary thereto, said sequence comprising at least one labelling means,
and which has a nucleic acid sequence substantially complementary to at least one part of the nucleic acid sequence amplified,
reagents necessary for carrying out an amplification reaction.

17. Kit according to claim 16, wherein the reagents necessary for carrying out an amplification reaction are reagents for a NASBA amplification.
